SEQUENCE LISTING (2 sequences)

SEQ ID NO: 1 - protein, 228 aa, Magnaporthe grisea
MQFITVALTLIALASASPIATNVEKPSELEARQLNSVRNDLISGNAAACPSVILIFARASGEVGNMGLSAGTNVASALEREFRNDIWVQGVGDPYDAALSPNFLPAGTTQGAIDEAKRMFTLANTKCPNAAVVAGGYSQGTAVMFNAVSEMPAAVQDQIKGVVLFGYTKNLQNRGRIPDFPTEKTEVYCNASDAVCFGTLFLLPAHFLYTTESSIAAPNWLIRQIRAA

SEQ ID NO: 2 - DNA, 5229 bp, Artificial Sequence
  Nucleotide sequence of expression cassette containing M. grisea cutinase gene used to transform T. reesei
aggcctctagagttgtgaagtcggtaatcccgctgtatagtaatacgagtcgcatctaaatactccgaagctgctgcgaacccggagaatcgagatgtgctggaaagcttctagcgagcggctaaattagcatgaaaggctatgagaaattctggagacggcttgttgaatcatggcgttccattcttcgacaagcaaagcgttccgtcgcagtagcaggcactcattcccgaaaaaactcggagattcctaagtagcgatggaaccggaataatataataggcaatacattgagttgcctcgacggttgcaatgcaggggtactgagcttggacataactgttccgtaccccacctcttctcaacctttggcgtttccctgattcagcgtacccgtacaagtcgtaatcactattaacccagactgaccggacgtgttttgcccttcatttggagaaataatgtcattgcgatgtgtaatttgcctgcttgaccgactggggctgttcgaagcccgaatgtaggattgttatccgaactctgctcgtagaggcatgttgtgaatctgtgtcgggcaggacacgcctcgaaggttcacggcaagggaaaccaccgatagcagtgtctagtagcaacctgtaaagccgcaatgcagcatcactggaaaatacaaaccaatggctaaaagtacataagttaatgcctaaagaagtcatataccagcggctaataattgtacaatcaagtggctaaacgtaccgtaatttgccaacggcttgtggggttgcagaagcaacggcaaagccccacttccccacgtttgtttcttcactcagtccaatctcagctggtgatcccccaattgggtcgcttgtttgttccggtgaagtgaaagaagacagaggtaagaatgtctgactcggagcgttttgcatacaaccaagggcagtgatggaagacagtgaaatgttgacattcaaggagtatttagccagggatgcttgagtgtatcgtgtaaggaggtttgtctgccgatacgacgaatactgtatagtcacttctgatgaagtggtccatattgaaatgtaaagtcggcactgaacaggcaaaagattgagttgaaactgcctaagatctcgggccctcgggccttcggcctttgggtgtacatgtttgtgctccgggcaaatgcaaagtgtggtaggatcgaacacactgctgcctttaccaagcagctgagggtatgtgataggcaaatgttcaggggccactgcatggtttcgaatagaaagagaagcttagccaagaacaatagccgataaagatagcctcattaaacggaatgagctagtaggcaaagtcagcgaatgtgtatatataaaggttcgaggtccgtgcctccctcatgctctccccatctactcatcaactcagatcctccaggagacttgtacaccatcttttgaggcacagaaacccaatagtcaaccatcacaagtttgtacaaaaaagcaggctatgcagtttattaccgtcgctttgacattgattgccttggcttccgcctcgcctattgccactaacgtggagaagccctctgagctggaagctcgccagcttaactcggtccgcaatgacctgatcagcggcaacgcagctgcctgcccctcggtcattctgatcttcgcccgcgcctctggtgaggtcggaaacatgggactctccgccggtaccaacgtcgcctcggctttggagcgtgagtttcgcaacgatatttgggtccagggcgtcggcgatccttacgacgccgctctctcaccaaacttcctgcctgctggcaccacccagggcgccatcgatgaggccaagcgcatgttcacactggccaacacgaagtgccccaacgctgctgttgttgccggcggatactcgcagggcacggccgttatgttcaacgcggttagcgagatgcccgcagctgtgcaggaccagatcaagggagtggttctgttcggctacacgaagaacctccagaaccgcggacgcatcccggactttccgaccgaaaagactgaggtctactgcaacgcttcggacgcggtgtgctttggtacgctgttcctccttcctgcgcacttcctctatactaccgagtcgagcatcgccgctccgaactggttgattcgtcaaatccgcgctgcgtaatgagatctacccagctttcttgtacaaagtggtgatcgcgccgcgcgccagctccgtgcgaaagcctgacgcaccggtagattcttggtgagcccgtatcatgacggcggcgggagctacatggccccgggtgatttattttttttgtatctacttctgacccttttcaaatatacggtcaactcatctttcactggagatgcggcctgcttggtattgcgatgttgtcagcttggcaaattgtggctttcgaaaacacaaaacgattccttagtagccatgcattttaagataacggaatagaagaaagaggaaattaaaaaaaaaaaaaaaacaaacatcccgttcataacccgtagaatcgccgctcttcgtgtatcccagtaccagtttattttgaatagctcgcccgctggagagcatcctgaatgcaagtaacaaccgtagaggctgacacggcaggtgttgctagggagcgtcgtgttctacaaggccagacgtcttcgcggttgatatatatgtatgtttgactgcaggctgctcagcgacgacagtcaagttcgccctcgctgcttgtgcaataatcgcagtggggaagccacaccgtgactcccatctttcagtaaagctctgttggtgtttatcagcaatacacgtaatttaaactcgttagcatggggctgatagcttaattaccgtttaccagtgccgcggttctgcagctttccttggcccgtaaaattcggcgaagccagccaatcaccagctaggcaccagctaaaccctataattagtctcttatcaacaccatccgctcccccgggatcaatgaggagaatgagggggatgcggggctaaagaagcctacataaccctcatgccaactcccagtttacactcgtcgagccaacatcctgactataagctaacacagaatgcctcaatcctgggaagaactggccgctgataagcgcgcccgcctcgcaaaaaccatccctgatgaatggaaagtccagacgctgcctgcggaagacagcgttattgatttcccaaagaaatcggggatcctttcagaggccgaactgaagatcacagaggcctccgctgcagatcttgtgtccaagctggcggccggagagttgacctcggtggaagttacgctagcattctgtaaacgggcagcaatcgcccagcagttagtagggtcccctctacctctcagggagatgtaacaacgccaccttatgggactatcaagctgacgctggcttctgtgcagacaaactgcgcccacgagttcttccctgacgccgctctcgcgcaggcaagggaactcgatgaatactacgcaaagcacaagagacccgttggtccactccatggcctccccatctctctcaaagaccagcttcgagtcaaggtacaccgttgcccctaagtcgttagatgtccctttttgtcagctaacatatgccaccagggctacgaaacatcaatgggctacatctcatggctaaacaagtacgacgaaggggactcggttctgacaaccatgctccgcaaagccggtgccgtcttctacgtcaagacctctgtcccgcagaccctgatggtctgcgagacagtcaacaacatcatcgggcgcaccgtcaacccacgcaacaagaactggtcgtgcggcggcagttctggtggtgagggtgcgatcgttgggattcgtggtggcgtcatcggtgtaggaacggatatcggtggctcgattcgagtgccggccgcgttcaacttcctgtacggtctaaggccgagtcatgggcggctgccgtatgcaaagatggcgaacagcatggagggtcaggagacggtgcacagcgttgtcgggccgattacgcactctgttgagggtgagtccttcgcctcttccttcttttcctgctctataccaggcctccactgtcctcctttcttgctttttatactatatacgagaccggcagtcactgatgaagtatgttagacctccgcctcttcaccaaatccgtcctcggtcaggagccatggaaatacgactccaaggtcatccccatgccctggcgccagtccgagtcggacattattgcctccaagatcaagaacggcgggctcaatatcggctactacaacttcgacggcaatgtccttccacaccctcctatcctgcgcggcgtggaaaccaccgtcgccgcactcgccaaagccggtcacaccgtgaccccgtggacgccatacaagcacgatttcggccacgatctcatctcccatatctacgcggctgacggcagcgccgacgtaatgcgcgatatcagtgcatccggcgagccggcgattccaaatatcaaagacctactgaacccgaacatcaaagctgttaacatgaacgagctctgggacacgcatctccagaagtggaattaccagatggagtaccttgagaaatggcgggaggctgaagaaaaggccgggaaggaactggacgccatcatcgcgccgattacgcctaccgctgcggtacggcatgaccagttccggtactatgggtatgcctctgtgatcaacctgctggatttcacgagcgtggttgttccggttacctttgcggataagaacatcgataagaagaatgagagtttcaaggcggttagtgagcttgatgccctcgtgcaggaagagtatgatccggaggcgtaccatggggcaccggttgcagtgcaggttatcggacggagactcagtgaagagaggacgttggcgattgcagaggaagtggggaagttgctgggaaatgtggtgactccatagctaataagtgtcagatagcaatttgcacaagaaatcaataccagcaactgtaaataagcgctgaagtgaccatgccatgctacgaaagagcagaaaaaaacctgccgtagaaccgaagagatatgacacgcttccatctctcaaaggaagaatcccttcagggttgcgtttccagtctagaggccattt

(19) United States
(12) Patent Application Publication
(10) Pub. No.: US 2015/0044736 A1
(43) Pub. Date: Feb. 12, 2015

(54) FUNGAL CUTINASE FROM MAGNAPORTHE GRISEA

(71) Applicant: Danisco US Inc., Palo Alto, CA (US)

(72) Inventors: Christian D. Adams, San Francisco, CA (US); Andrei Miasnikov, Mountain View, CA (US)

(73) Assignee: DANISCO US INC., Palo Alto, CA (US)

(21) Appl. No.: 14/338,639

(22) Filed: Jul. 23, 2014

Related U.S. Application Data
(63) Continuation of application No. 13/257,235, filed on Sep. 16, 2011, now abandoned.
(60) Provisional application No. 61/161,175, filed on Mar. 18, 2009.

(51) Int. Cl. C12N 9/18; C11D 3/386; C12P 7/64

(52) U.S. Cl. CPC C12N 9/18; C12P 7/6454; C11D 3/38627; C12Y 301/01074
USPC 435/134; 435/197; 435/264; 510/392

(57) ABSTRACT

Described are compositions and methods relating to a fungal cutinase cloned from Magnaporthe grisea, polynucleotides encoding the cutinase, and methods of use thereof. The compositions and methods have particular application in detergent cleaning compositions and synthesis reactions.

### PRIORITY

The present application is a continuation application of U.S. patent application Ser. No. 13/257,235, filed Sep. 16, 2011, which is a U.S. National Stage Application of International Application No. PCT/US2010/025254, filed Feb. 24, 2010, which claims priority to U.S. Provisional Patent Application Ser. No. 61/161,175, filed on Mar. 18, 2009, which are hereby incorporated by reference in their entirety.

### TECHNICAL FIELD

The present compositions and methods relate to a fungal cutinase cloned from Magnaporthe grisea, polynucleotides encoding the cutinase, and methods of use thereof.

### BACKGROUND

Current laundry detergent and/or fabric care compositions include a complex combination of active ingredients such as surfactants, enzymes (protease, amylase, lipase, and/or cellulase), bleaching agents, a builder system, suds suppressors, soil-suspending agents, soil-release agents, optical brighteners, softening agents, dispersants, dye transfer inhibition compounds, abrasives, bactericides, and perfumes.

Lipolytic enzymes, including lipases and cutinases, have been employed in detergent cleaning compositions for the removal of oily stains by hydrolyzing triglycerides to generate fatty acids. However, these enzymes are often inhibited by surfactants and other components present in cleaning composition, interfering with their ability to remove oily stains. Accordingly, the need exists for lipases and cutinases that can function in the harsh environment of cleaning compositions.

There also exists a need for more robust and efficient lipases and cutinases that are effective in performing transesterification reactions for the production of biofuels, lubricants, and other synthetic and semi-synthetic hydrocarbons. Preferably, such enzymes will utilize naturally occurring or commonly available starting materials and will not require protection and deprotection steps in a synthesis reaction, which complicate the synthesis and lead to the production of toxic waste products.

### SUMMARY

The present compositions and methods relate to a fungal cutinase cloned from Magnaporthe grisea, also known as Pyricularia grisea or rice blast fungus.

In one aspect, a recombinant Mgr-C polypeptide having an amino acid sequence that is at least 80% identical to the amino acid sequence of SEQ ID NO: 1 is provided. In some embodiments, the recombinant Mgr-C polypeptide is at least 90% identical to the amino acid sequence of SEQ ID NO: 1. In some embodiments, the recombinant Mgr-C polypeptide is substantially identical to the amino acid sequence of SEQ ID NO: 1. In particular embodiments, the recombinant Mgr-C polypeptide has the amino acid sequence of SEQ ID NO: 1.

In some embodiments, the polypeptide is expressed in a heterologous organism as a secreted polypeptide. In some embodiments, the organism is a filamentous fungus. In particular embodiments, the organism is Trichoderma reesei.

In another aspect, an expression vector comprising a polynucleotide encoding an Mgr-C polypeptide is provided, the polypeptide having an amino acid sequence that is at least 80% identical to the amino acid sequence of SEQ ID NO: 1 operably linked to a signal sequence for directing the secretion of the Mgr-C polypeptide. In some embodiments, the polynucleotide encodes an Mgr-C polypeptide having an amino acid sequence that is at least 90% identical to the amino acid sequence of SEQ ID NO: 1. In some embodiments, the polynucleotide encodes an Mgr-C polypeptide having an amino acid sequence that is substantially identical to amino acid sequence of SEQ ID NO: 1. In particular embodiments, the polynucleotide encodes an Mgr-C polypeptide having the amino acid sequence of SEQ ID NO: 1. In some embodiments, the signal sequence is from Trichoderma reesei.

In another aspect, a detergent composition comprising an Mgr-C polypeptide is provided. In some embodiments, the composition comprises a non-ionic surfactant. In particular embodiments, the surfactant is non-ionic ethoxylate surfactant.

In another aspect, a method for hydrolyzing a lipid present in a soil or stain on a surface is provided, comprising contacting the surface with a detergent composition comprising a recombinant Mgr-C polypeptide and a non-ionic surfactant. In some embodiments, the non-ionic surfactant is an ethoxylate surfactant.

In another aspect, a method for performing a transesterification reaction is provided, comprising contacting a donor molecule with a composition comprising a recombinant Mgr-C polypeptide. In some embodiments, the donor molecule has a C4-C10 carbon chain. In some embodiments, the donor molecule has a C8 carbon chain. In some embodiments, the Mgr-C polypeptide has an amino acid sequence that is at least 80% identical to the amino acid sequence of SEQ ID NO: 1. In some embodiments, the Mgr-C polypeptide has an amino acid sequence that is at least 90% identical to the amino acid sequence of SEQ ID NO: 1. In some embodiments, the Mgr-C polypeptide has an amino acid that is substantially identical to amino acid sequence of SEQ ID NO: 1. In particular embodiments, the Mgr-C polypeptide has the amino acid sequence of SEQ ID NO: 1.

These and other aspects of Mgr-C compositions and methods will be apparent from the following description and drawings.

### BRIEF DESCRIPTION OF THE FIGURES

FIG. 1 is a diagram of an expression vector used to express an Mgr-C polypeptide in Trichoderma reesei.

FIG. 2 is an image of a Coomassie-stained SDS-polyacrylamide gel showing expression of Mgr-C.

FIG. 3 is a graph showing the pH activity profile of Mgr-C.

FIG. 4 is a graph showing the temperature activity profile of Mgr-C.

FIG. 5 is a graph showing the chain-length preference of Mgr-C.

FIG. 6 is a graph showing the hydrolysis of trioctanoate by Mgr-C in solution.

FIGS. 7A and 7B are graphs showing the hydrolysis of trioctanoate by Mgr-C on cloth as measured by the NEFA assay. (A) Free fatty acids remaining on cloth. (B) Free fatty acids released into solution

FIG. 8A is a diagram showing the structure of polyester having terephthalate monomers (shown in a box). FIG. 8B is a bar graph showing Mgr-C polyesterase activity

FIG. 9 is a bar graph showing stain removal performance Mgr-C measured by a Lard/Sudan Red assay.

### DETAILED DESCRIPTION

### I. Introduction

Described are compositions and methods relating to a fungal cutinase cloned from Magnaporthe grisea (Pyricularia grisea or rice blast fungus). The compositions and methods are based, in part, on the observation that cloned and expressed Mgr-C has carboxylic ester hydrolase activity, and that Mgr-C is active in the presence of a detergent composition. These features of Mgr-C make it well suited a variety of cleaning applications, where the enzyme can hydrolyze lipids in the presence of surfactants and other components found in detergent compositions.

While Mgr-C showed activity against a variety of natural and synthetic substrates used to evaluate triglyceride hydrolysis, the enzyme had a preference for C4-C10 substrates, with peak activity against C8 substrates. This specificity make Mgr-C well suited for hydrolysis of short-chain triglycerides and for performing transesterification reactions involving short-chain fatty acids.

These and other features of Mgr-C are to be described.

### II. Definitions

Prior to describing the present compositions and methods in detail, the following terms are defined for clarity. Terms and abbreviations not defined should be accorded their ordinary meaning as used in the art:

As used herein, a “a carboxylic ester hydrolase” (E.C. 3.1.1) refers to an enzyme that acts on carboxylic acid esters. One type of carboxylic ester hydrolase is a “cutinase,” which degrades the waxy protective surface polymer (cutin) of aerial parts of plants. Cutinases generally fall under several enzyme classifications, including but not limited to E.C. 3.1.1.50 and EC 3.1.1.74. However, cutinases, and carboxylic ester hydrolases, generally, are often capable of catalyzing a broad range of reactions, e.g., lipase, esterase, transesterase, acyltransferase, and similar related reactions, which may involve substrates and activities other than those which led to their particular enzyme classification. Therefore, it will be appreciate that the use of the term cutinase, herein, is not intended to define substrate specificity or activity but only to describe the present polypeptides in terms of their structural relatedness to known molecules.

As used herein, the term “fatty acid” refers to a carboxylic acid derived from or contained in an animal or vegetable fat or oil. Fatty acids are composed of a chain of alkyl groups typically containing from 4-22 carbon atoms and characterized by a terminal carboxyl group (—COOH). Fatty acids may be saturated or unsaturated, and solid, semisolid, or liquid.

As used herein, the term “triglyceride” refers to any naturally occurring ester of a fatty acid and glycerol. Triglycerides are the chief constituents of fats and oils. The have the general formula of CH₂(OOCROCH(OOCR₂)CH₂(OOCR₃), where R₁, R₂, and R₃ may be of different chain length.

As used herein, “acyl” is the general name for an organic acid group (RCO—), generally obtained by removing the —OH group from a carboxylic acid.

As used herein, the term “acylation” refers to a chemical transformation which substitutes/adds an acyl group into a molecule, generally at the side of an —OH group.

As used herein, an “acyl chain substrate” is a donor molecule for a carboxylic ester hydrolase (e.g., cutinase, lipase, acyltransferase, transesterase, and the like). The substrate may be described in terms of its carbon-chain length. For example, a C4 substrate/donor has a chain-length of 4 carbons, a C8 substrate/donor has a chain-length of 8 carbons, and the like.

As used herein, the term “transferase” refers to an enzyme that catalyzes the transfer of a molecule or group (e.g., an acyl group) to a substrate.

As used herein, “leaving group” refers to the nucleophile which is cleaved from the acyl donor upon substitution by another nucleophile.

As used herein, the phrase “detergent stability” refers to the stability of a specified detergent composition component (such as a hydrolytic enzyme) in a detergent composition mixture.

As used herein, a “perhydrolase” is an enzyme capable of catalyzing a reaction that results in the formation of a peracid suitable for applications such as cleaning, bleaching, and disinfecting.

As used herein, the term “aqueous,” as used in the phrases “aqueous composition” and “aqueous environment,” refers to a composition that is made up of at least 50% water. An aqueous composition may contain at least 50% water, at least 60% water, at least 70% water, at least 80% water, at least 90% water, at least 95% water, at least 97% water, at least 99% water, or even at least 99% water.

As used herein, the term “surfactant” refers to any compound generally recognized in the art as having surface active qualities. Surfactants generally include anionic, cationic, nonionic, and zwitterionic compounds, which are further described, herein.

As used herein, “cleaning compositions” and “cleaning formulations” refer to admixtures of chemical ingredients that find use in the removal of undesired compounds (e.g., soil or stains) from items to be cleaned, such as fabric, dishes, contact lenses, other solid surfaces, hair, skin, teeth, and the like. The composition or formulations may be in the form of a liquid, gel, granule, powder, or spray, depending on the surface, item or fabric to be cleaned, and the desired form of the composition or formulation.

As used herein, the terms “detergent composition” and “detergent formulation” refer to mixtures of chemical ingredients intended for use in a wash medium for the cleaning of soiled objects. Detergent compositions/formulations generally include at least one surfactant, and may optionally include hydrolytic enzymes, oxido-reductases, builders, bleaching agents, bleach activators, bluing agents and fluorescent dyes, caking inhibitors, masking agents, enzyme activators, antioxidants, and solubilizers.

As used herein, the terms “textile” or “textile material” refer to woven fabrics, as well as staple fibers and filaments suitable for conversion to or use as yarns, woven, knit, and non-woven fabrics. The term encompasses yarns made from natural, as well as synthetic (e.g., manufactured) fibers.

As used herein, the terms “purified” and “isolated” refer to the physical separation of a subject molecule, such as Mgr-C cutinase, from other molecules, such as proteins, nucleic acids, lipids, media components, and the like. Once purified or isolated, a subject molecule may represent at least 50%, and even at least 60%, at least 70%, at least 80%, at least 85%, at least 90%, at least 95%, or more, of the total amount of material in a sample (wt/wt).

As used herein, a “polypeptide” refers to a molecule comprising a plurality of amino acids linked through peptide bonds. The terms “polypeptide,” “peptide,” and “protein” are used interchangeably. Proteins maybe optionally be modified (e.g., glycosylated, phosphorylated, acylated, farnesylated, prenylated, sulfonated, and the like) to add functionality. Where such amino acid sequences exhibit activity, they may be referred to as an “enzyme.” The conventional one-letter or three-letter codes for amino acid residues are used, with amino acid sequences being presented in the standard amino-to-carboxy terminal orientation (i.e., N→C).

The terms “polynucleotide” encompasses DNA, RNA, heteroduplexes, and synthetic molecules capable of encoding a polypeptide. Nucleic acids may be single stranded or double stranded, and may be chemical modifications. The terms “nucleic acid” and “polynucleotide” are used interchangeably. Because the genetic code is degenerate, more than one codon may be used to encode a particular amino acid, and the present compositions and methods encompass nucleotide sequences which encode a particular amino acid sequence. Unless otherwise indicated, nucleic acid sequences are presented in a 5′-to-3′ orientation.

As used herein, the terms “wild-type” and “native” refer to polypeptides or polynucleotides that are found in nature.

The terms, “wild-type,” “parental,” or “reference,” with respect to a polypeptide, refer to a naturally-occurring polypeptide that does not include a man-made substitution, insertion, or deletion at one or more amino acid positions. Similarly, the terms “wild-type,” “parental,” or “reference,” with respect to a polynucleotide, refer to a naturally-occurring polynucleotide that does not include a man-made nucleoside change. However, note that a polynucleotide encoding a wild-type, parental, or reference polypeptide is not limited to a naturally-occurring polynucleotide, and encompasses any polynucleotide encoding the wild-type, parental, or reference polypeptide.

As used herein, a “variant polypeptide” refers to a polypeptide that is derived from a parent (or reference) polypeptide by the substitution, addition, or deletion, of one or more amino acids, typically by recombinant DNA techniques. Variant polypeptides may differ from a parent polypeptide by a small number of amino acid residues and may be defined by their level of primary amino acid sequence homology/identity with a parent polypeptide. Preferably, variant polypeptides have at least 70%, at least 75%, at least 80%, at least 85%, at least 90%, at least 91%, at least 92%, at least 93%, at least 94%, at least 95%, at least 96%, at least 97%, at least 98%, or even at least 99% amino acid sequence identity with a parent polypeptide.

Sequence identity may be determined using known programs such as BLAST, ALIGN, and CLUSTAL using standard parameters. (See, e.g., Altschul et al. (1990) J. Mol. Biol. 215:403-410; Henikoff et al. (1989) Proc. Natl. Acad. Sci. USA 89:10915; Karin et al. (1993) Proc. Natl. Acad. Sci USA 90:5873; and Higgins et al. (1988) Gene 73:237-244). Software for performing BLAST analyses is publicly available through the National Center for Biotechnology Information. Also, databases may be searched using FASTA (Pearson et al. (1988) Proc. Natl. Acad. Sci. USA 85:2444-2448). One indication that two polypeptides are substantially identical is that the first polypeptide is immunologically cross-reactive with the second polypeptide. Typically, polypeptides that differ by conservative amino acid substitutions are immunologically cross-reactive. Thus, a polypeptide is substantially identical to a second polypeptide, for example, where the two peptides differ only by a conservative substitution.

As used herein, a “variant polynucleotide” encodes a variant polypeptide, has a specified degree of homology/identity with a parent polynucleotide, or hybridized under stringent conditions to a parent polynucleotide or the complement, thereof. Preferably, a variant polynucleotide has at least 70%, at least 75%, at least 80%, at least 85%, at least 90%, at least 91%, at least 92%, at least 93%, at least 94%, at least 95%, at least 96%, at least 97%, at least 98%, or even at least 99% nucleotide sequence identity with a parent polynucleotide. Methods for determining percent identity are known in the art and described immediately above.

The term “derived from” encompasses the terms “originated from,” “obtained from,” “obtainable from,” “isolated from,” and “created from,” and generally indicates that one specified material find its origin in another specified material or has features that can be described with reference to the another specified material.

As used herein, the term “hybridization” refers to the process by which a strand of nucleic acid joins with a complementary strand through base pairing, as known in the art

As used herein, the phrase “hybridization conditions” refers to the conditions under which hybridization reactions are conducted. These conditions are typically classified by degree of “stringency” of the conditions under which hybridization is measured. The degree of stringency can be based, for example, on the melting temperature (Tm) of the nucleic acid binding complex or probe. For example, “maximum stringency” typically occurs at about Tm-5° C. (5° below the Tm of the probe); “high stringency” at about 5-10° below the Tm; “intermediate stringency” at about 10-20° below the Tm of the probe; and “low stringency” at about 20-25° below the Tm. Alternatively, or in addition, hybridization conditions can be based upon the salt or ionic strength conditions of hybridization and/or one or more stringency washes, e.g.: 6×SSC=very low stringency; 3×SSC=low to medium stringency; 1×SSC=medium stringency; and 0.5×SSC=high stringency. Functionally, maximum stringency conditions may be used to identify nucleic acid sequences having strict identity or near-strict identity with the hybridization probe; while high stringency conditions are used to identify nucleic acid sequences having about 80% or more sequence identity with the probe. For applications requiring high selectivity, it is typically desirable to use relatively stringent conditions to form the hybrids (e.g., relatively low salt and/or high temperature conditions are used). As used herein, stringent conditions are defined as 50° C. and 0.2×SSC (1×SSC=0.15 M NaCl, 0.015 M sodium citrate, pH 7.0).

The phrases “substantially similar and “substantially identical” in the context of at least two nucleic acids or polypeptides means that a polynucleotide or polypeptide comprises a sequence that has at least about 90%, at least about 91%, at least about 92%, at least about 93%, at least about 94%, at least about 95%, at least about 96%, at least about 97%, at least about 98%, or even at least about 99% identical to a parent or reference sequence, or does not include amino acid substitutions, insertions, deletions, or modifications made only to circumvent the present description without adding functionality.

As used herein, an “expression vector” refers to a DNA construct containing a DNA sequence that encodes a specified polypeptide and is operably linked to a suitable control sequence capable of effecting the expression of the polypeptides in a suitable host. Such control sequences include a promoter to effect transcription, an optional operator sequence to control such transcription, a sequence encoding suitable mRNA ribosome binding sites and sequences which control termination of transcription and translation. The vector may be a plasmid, a phage particle, or simply a potential genomic insert. Once transformed into a suitable host, the vector may replicate and function independently of the host genome, or may, in some instances, integrate into the genome itself.

The term “recombinant,” refers to genetic material (i.e., nucleic acids, the polypeptides they encode, and vectors and cells comprising such polynucleotides) that has been modified to alter its sequence or expression characteristics, such as by mutating the coding sequence to produce an altered polypeptide, fusing the coding sequence to that of another gene, placing a gene under the control of a different promoter, expressing a gene in a heterologous organism, expressing a gene at a decreased or elevated levels, expressing a gene conditionally or constitutively in manner different from its natural expression profile, and the like. Generally recombinant nucleic acids, polypeptides, and cells based thereon, have been manipulated by man such that they are not identical to related nucleic acids, polypeptides, and cells found in nature.

A “signal sequence” refers to a sequence of amino acids bound to the N-terminal portion of a polypeptide, and which facilitates the secretion of the mature form of the protein from the cell. The mature form of the extracellular protein lacks the signal sequence which is cleaved off during the secretion process.

The term “selective marker” or “selectable marker” refers to a gene capable of expression in a host cell that allows for ease of selection of those hosts containing an introduced nucleic acid or vector. Examples of selectable markers include but are not limited to antimicrobial substances (e.g., hygromycin, bleomycin, or chloramphenicol) and/or genes that confer a metabolic advantage, such as a nutritional advantage, on the host cell.

The term “regulatory element” as used herein refers to a genetic element that controls some aspect of the expression of nucleic acid sequences. For example, a promoter is a regulatory element which facilitates the initiation of transcription of an operably linked coding region. Additional regulatory elements include splicing signals, polyadenylation signals and termination signals.

As used herein, “host cells” are generally prokaryotic or eukaryotic hosts which are transformed or transfected with vectors constructed using recombinant DNA techniques known in the art. Transformed host cells are capable of either replicating vectors encoding the protein variants or expressing the desired protein variant. In the case of vectors which encode the pre- or prepro-form of the protein variant, such variants, when expressed, are typically secreted from the host cell into the host cell medium.

The term “introduced” in the context of inserting a nucleic acid sequence into a cell, means transformation, transduction or transfection. Means of transformation include protoplast transformation, calcium chloride precipitation, electroporation, naked DNA and the like as known in the art. (See, Chang and Cohen (1979) Mol. Gen. Genet., 168:111-115; Smith et al. (1986) Appl. Env. Microbiol., 51:634; and the review article by Ferrari et al., in Harwood, Bacillus, Plenum Publishing Corporation, pp. 57-72, 1989).

The terms “selectable marker” or “selectable gene product” as used herein refer to the use of a gene which encodes an enzymatic activity that confers resistance to an antibiotic or drug upon the cell in which the selectable marker is expressed.

The following abbreviations apply: ° C. (degrees Centigrade); rpm (revolutions per minute); H₂O (water); HCl (hydrochloric acid); aa (amino acid); by (base pair); kb (kilobase pair); kD (kilodalton); g, gm (gram); μg and ug (microgram); mg (milligram); ng (nanogram); μl and ul (microliter); ml (milliliter); mm (millimeter); nm (nanometer); μm and um (micrometer); M (molar); mM (millimolar); μM and uM (micromolar); U (unit); V (volt); MW (molecular weight); sec (second); min(s) (minute/minutes); hr(s) (hour/hours); MgCl₂ (magnesium chloride); NaCl (sodium chloride); OD₂₈₀ (optical density at 280 nm); OD₆₀₀ (optical density at 600 nm); PAGE (polyacrylamide gel electrophoresis); EtOH (ethanol); PBS (phosphate buffered saline [150 mM NaCl, 10 mM sodium phosphate buffer, pH 7.2]); SDS (sodium dodecyl sulfate); Tris (tris(hydroxymethyl)aminomethane); TAED (N,N,N,′N′-tetraacetylethylenediamine); w/v (weight to volume); v/v (volume to volume); MS (mass spectroscopy); p (para); m (meta); o (ortho).

Other technical and scientific have the same meaning as commonly understood by one of ordinary skill in the art to which this invention pertains (see, e.g., Singleton and Sainsbury, Dictionary of Microbiology and Molecular Biology, 2d Ed., John Wiley and Sons, NY (1994); and Hale and Marham, The Harper Collins Dictionary of Biology, Harper Perennial, N.Y. (1991).

The singular terms “a,” “an,” and “the” include the plural reference unless the context clearly indicates otherwise.

Headings are provided for convenience and should not be construed as limitations. The description included under one heading may apply to the specification as a whole.

### III. Mgr-C Polypeptides and Polynucleotides

A. Mgr-C Polypeptides

In one aspect, the present compositions and methods provide a recombinant Mgr-C polypeptide or a variant thereof. An exemplary Mgr-C polypeptide was isolated from Magnaporthe grisea, also known as Pyricularia grisea or rice blast fungus, and has the amino acid sequence of SEQ ID NO: 2. Similar, substantially identical Mgr-C polypeptides may occur in nature, e.g., in other strains or isolates of M. grisea or closely related fungi. For example, Sweigard, J. A. et al. ((1992) Mol. Gen. Genet. 232:174-82) described a polypeptide that differs from the exemplary Mgr-C polypeptide by the presence of an Arg at position 61 of the mature polypeptide, rather than an Ala. These and other recombinant MgrC polypeptides are encompassed by the present compositions and methods.

In addition to recombinant MgrC polypeptides from M. grisea, the composition and methods include recombinant Mgr-C polypeptides from other fungi, such as members of the Magnaporthaceae family, including, Buergenerula spp., Ceratosphaeria spp., Gaeumannomyces spp., Juncigena spp., Ophioceras spp., Pseudohalonectria spp., Magnaporthaceae spp., and other Magnaporthe spp.

In some embodiments, the recombinant Mgr-C polypeptide is a variant Mgr-C polypeptide having a specified degree of amino acid sequence homology to the exemplified Mgr-C polypeptide, e.g., at least 70%, at least 75%, at least 80%, at least 85%, at least 90%, at least 91%, at least 92%, at least 93%, at least 94%, at least 95%, at least 96%, at least 97%, at least 98%, or even at least 99% sequence homology to the amino acid sequence of SEQ ID NO: 2. Homology can be determined by amino acid sequence alignment, e.g., using a program such as BLAST, ALIGN, or CLUSTAL, as described herein.

In some embodiments, the recombinant Mgr-C polypeptide includes substitutions that do not substantially affect the structure and/or function of the polypeptide. Exemplary substitutions are conservative mutations, as summarized in the following Table.

| Original Amino | | |
|---|---|---|
| Acid Residue | Code | Acceptable Substitutions |
| Alanine | A | D-Ala, Gly, beta-Ala, L-Cys, D-Cys |
| Arginine | R | D-Arg, Lys, D-Lys, homo-Arg, D-homo-Arg, |
| | | Met, Ile, D-Met, D-Ile, Orn, D-Orn |
| Asparagine | N | D-Asn, Asp, D-Asp, Glu, D-Glu, Gln, D-Gln |
| Aspartic Acid | D | D-Asp, D-Asn, Asn, Glu, D-Glu, Gln, D-Gln |
| Cysteine | C | D-Cys, S-Me-Cys, Met, D-Met, Thr, D-Thr |
| Glutamine | Q | D-Gln, Asn, D-Asn, Glu, D-Glu, Asp, D-Asp |
| Glutamic Acid | E | D-Glu, D-Asp, Asp, Asn, D-Asn, Gln, D-Gln |
| Glycine | G | Ala, D-Ala, Pro, D-Pro, beta-Ala, Acp |
| Isoleucine | I | D-Ile, Val, D-Val, Leu, D-Leu, Met, D-Met |
| Leucine | L | D-Leu, Val, D-Val, Leu, D-Leu, Met, D-Met |
| Lysine | K | D-Lys, Arg, D-Arg, homo-Arg, D-homo-Arg, |
| | | Met, D-Met, Ile, D-Ile, Orn, D-Orn |
| Methionine | M | D-Met, S-Me-Cys, Ile, D-Ile, Leu, D-Leu, Val, |
| | | D-Val |
| Phenylalanine | F | D-Phe, Tyr, D-Thr, L-Dopa, His, D-His, Trp, |
| | | D-Trp, Trans-3,4, or 5-phenylproline, cis-3,4, |
| | | or 5-phenylproline |
| Proline | P | D-Pro, L-I-thioazolidine-4-carboxylic acid, D-or |
| | | L-1-oxazolidine-4-carboxylic acid |
| Serine | S | D-Ser, Thr, D-Thr, allo-Thr, Met, D-Met, |
| | | Met(O), D-Met(O), L-Cys, D-Cys |
| Threonine | T | D-Thr, Ser, D-Ser, allo-Thr, Met, |
| | | D-Met, Met(O), D-Met(O), Val, D-Val |
| Tyrosine | Y | D-Tyr, Phe, D-Phe, L-Dopa, His, D-His |
| Valine | V | D-Val, Leu, D-Leu, Ile, D-Ile, Met, D-Met |

Substitutions involving naturally occurring amino acids are generally made by mutating a nucleic acid encoding a recombinant Mgr-C polypeptide, and then expressing the variant polypeptide in an organism. Substitutions involving non-naturally occurring amino acids or chemical modifications to amino acids are generally made by chemically modifying a recombinant Mgr-C polypeptides after it has been synthesized by an organism.

In some embodiments, variant recombinant Mgr-C polypeptides are substantially identical to SEQ ID NO: 2, meaning that they do not include amino acid substitutions, insertions, or deletions that do not significantly affect the structure, function or expression of the polypeptide. Such variant recombinant Mgr-C polypeptides include those designed only to circumvent the present description.

In some embodiments, the recombinant Mgr-C polypeptide (including a variant, thereof) has carboxylic ester hydrolase activity, which includes lipase, esterase, transesterase, and/or acyltransferase activity. Carboxylic ester hydrolase activity can be determined and measured using the assays described herein, or by other assays known in the art. In some embodiments, the recombinant Mgr-C polypeptide has activity in the presence of a detergent composition.

Mgr-C polypeptides include fragments of “full-length” Mgr-C polypeptides that retain carboxylic ester hydrolase activity. Such fragments preferably retain the active site of the full-length polypeptides but may have deletions of non-critical amino acid residues. The activity of fragments can readily be determined using the assays described, herein, or by other assays known in the art. In some embodiments, the fragments of Mgr-C polypeptides retain carboxylic ester hydrolase activity in the presence of a detergent composition.

In some embodiments, the Mgr-C polypeptide is expressed in a heterologous organism, i.e., an organism other than Magnaporthe grisea. In some embodiments, the heterologous organism is not of the Magnaporthaceae family. Exemplary heterologous organisms are Gram(+) bacteria such as Bacillus subtilis, Bacillus licheniformis, Bacillus lentus, Bacillus brevis, Geobacillus (formerly Bacillus) stearothermophilus, Bacillus alkalophilus, Bacillus amyloliquefaciens, Bacillus coagulans, Bacillus circulans, Bacillus lautus, Bacillus megaterium, Bacillus thuringiensis, Streptomyces lividans, or Streptomyces murinus; Gram(−) bacteria such as E. coli.; yeast such as Saccharomyces spp. or Schizosaccharomyces spp., e.g. Saccharomyces cerevisiae; and filamentous fungi such as Aspergillus spp., e.g., Aspergillus oryzae or Aspergillus niger, and Trichoderma reesei. Methods from transforming nucleic acids into these organisms are well known in the art. A suitable procedure for transformation of Aspergillus host cells is described in EP 238 023.

In particular embodiments, the Mgr-C polypeptide is expressed in a heterologous organism as a secreted polypeptide, in which case, the compositions and method encompass a method for expressing an Mgr-C polypeptide as a secreted polypeptide in a heterologous organism.

B. Mgr-C Polynucleotides

Another aspect of the compositions and methods is a polynucleotide that encodes an Mgr-C polypeptide (including variants and fragments, thereof), provided in the context of an expression vector for directing the expression of an Mgr-C polypeptide in a heterologous organism, such as those identified, herein. The polynucleotide that encodes an Mgr-C polypeptide may be operably-linked to regulatory elements (e.g., a promoter, terminator, enhancer, and the like) to assist in expressing the encoded polypeptides.

In some embodiments, the polynucleotide that encodes an Mgr-C polypeptide is fused in frame behind (i.e., downstream of) a coding sequence for a signal peptide for directing the extracellular secretion of an Mgr-C polypeptide. Heterologous signal sequences include those from the Trichoderma reesei cbhl cellulase gene. An exemplary expression vector is described in Example 2. The polynucleotide may also be fused to a coding sequence for a different polypeptide, thereby encoding a chimeric polypeptide. Expression vectors may be provided in a heterologous host cell suitable for expressing an Mgr-C polypeptide, or suitable for propagating the expression vector prior to introducing it into a suitable host cell.

An exemplary polynucleotide sequence encoding an Mgr-C polypeptide has the nucleotide sequence of SEQ ID NO: 1. Similar, including substantially identical, polynucleotides encoding Mgr-C polypeptides and variants may occur in nature, e.g., in other strains or isolates of Magnaporthe grisea or closely related fungi, such as those described, above. In view of the degeneracy of the genetic code, it will be appreciated that polynucleotides having different nucleotide sequences may encode the same Mgr-C polypeptides, variants, or fragments.

In some embodiments, polynucleotides encoding Mgr-C polypeptides have a specified degree of amino acid sequence homology to the exemplified polynucleotide encoding an Mgr-C polypeptide, e.g., at least 70%, at least 75%, at least 80%, at least 85%, at least 90%, at least 91%, at least 92%, at least 93%, at least 94%, at least 95%, at least 96%, at least 97%, at least 98%, or even at least 99% sequence homology to the amino acid sequence of SEQ ID NO: 2. Homology can be determined by amino acid sequence alignment, e.g., using a program such as BLAST, ALIGN, or CLUSTAL, as described herein.

In some embodiments, polynucleotides encoding Mgr-C polypeptides hybridize to the exemplary polynucleotide of SEQ ID NO: 2 (or the complement, thereof) under specified hybridization conditions. Exemplary conditions are stringent condition and highly stringent conditions, which are described, herein.

Mgr-C polynucleotides may be naturally occurring or synthetic (i.e., man-made), and may be codon-optimized for expression in a different host, mutated to introduce cloning sites, or otherwise altered to add functionality.

### IV. Activities of Mgr-C

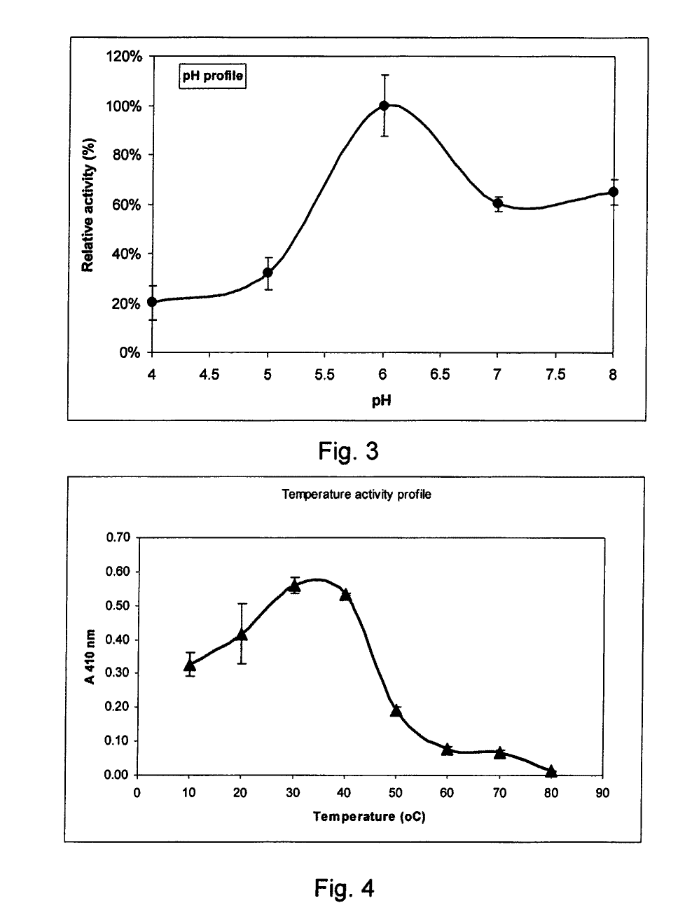

Experiments performed in support of the compositions and methods demonstrated that purified Mgr-C polypeptide, as well as crude and partially purified cellular material that contained Mgr-C polypeptide, demonstrated carboxylic ester hydrolase activity against natural and synthetic substrates. Activity against a synthetic substrate (pNO) was observed over the entire tested pH range tested, i.e., pH 4 to pH 8, with over 50% of maximum activity between about pH 5.5 and above (FIG. 3), suggesting that Mgr-C has activity over a broad range of pH conditions. Accordingly, exemplary pH ranges for Mgr-C are from about 4-8 and higher, from about 5.5-8, and from about 5.5-7.

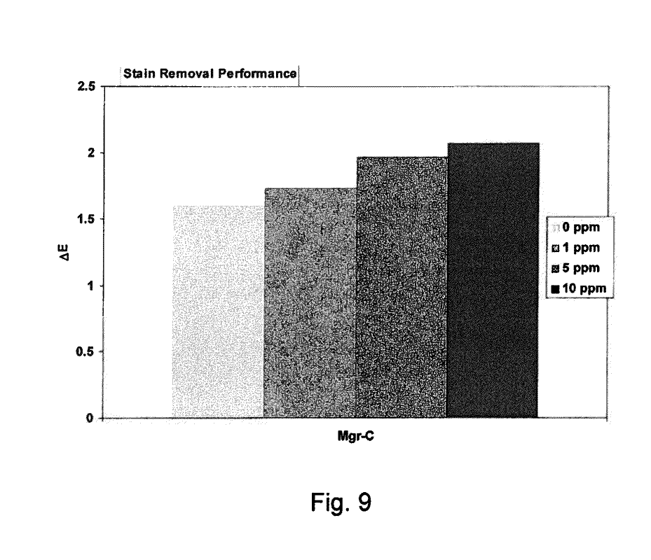

As shown in FIG. 4, Mgr-C is active over a wide range of temperatures, e.g., from 10° C. or lower to about 50° C. However, activity at 50° C. is substantially less that activity at 40° C. The optimum temperature range for Mgr-C is from about 30° C. to about 40° C., although the enzyme clearly has significant activity outside this range. The low temperature stain removal performance of Mgr-C was further demonstrated in a swatch cleaning assay performed at 20° C., as shown in FIG. 9. The ability of Mgr-C to function at low temperatures makes the enzyme well-suited for use in cold water cleaning applications.

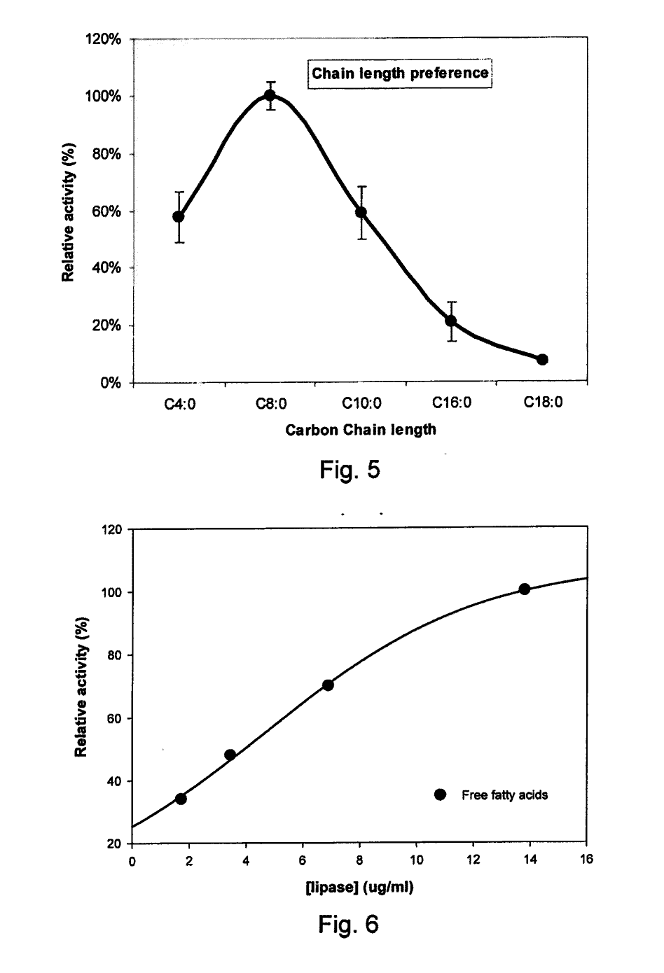

As shown in FIG. 5, the activity of Mgr-C was highest using a C8 substrate but a significant amount of activity was observed using C4 and C10 substrates. In contrast, the commercially product lipase LIPOMAX™ (Pseudomonas pseudoalcaligenes lipase variant M21L, Genencor Int. Inc., Palo Alto, Calif., USA) had a preference for C10 substrates, with activity falling off rapidly with smaller (e.g., C8) or larger (e.g., C16) substrates (not shown). Therefore, Mgr-C appear to be less selective that LIPOMAX for substrates of a particular length, while having a preference for substrates with a shorter chain length than LIPOMAX.

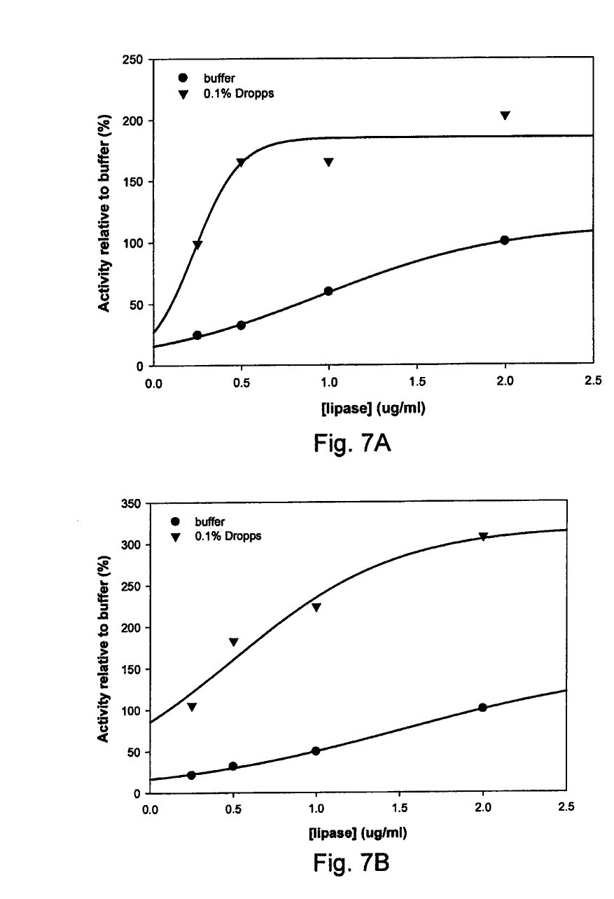

Mgr-C hydrolysis activity against an exemplary oily stain material, i.e., trioctanoate, was significantly enhanced in the presence of a surfactant compositions (i.e., DROPPS) both in solution (FIG. 6) and when trioctanoate was present on cloth (FIGS. 7A and 7B). In contrast, Mgr-C activity was reduced in the presence of another surfactant compositions (i.e., TIDE® CW; not shown). DROPPS is a detergent composition having only a non-ionic ethoxylate surfactant and very low water content (about 10% by weight). In contrast, TIDE has both anionic and nonionic surfactants and higher water content (about 30-40% by weight). Therefore, the preliminary data suggest that Mgr-C is active in the presence of nonionic surfactants and/or detergent composition with low water content, in which the enzyme is less soluble but not in anionic surfactants and/or and detergent compositions with a higher water content.

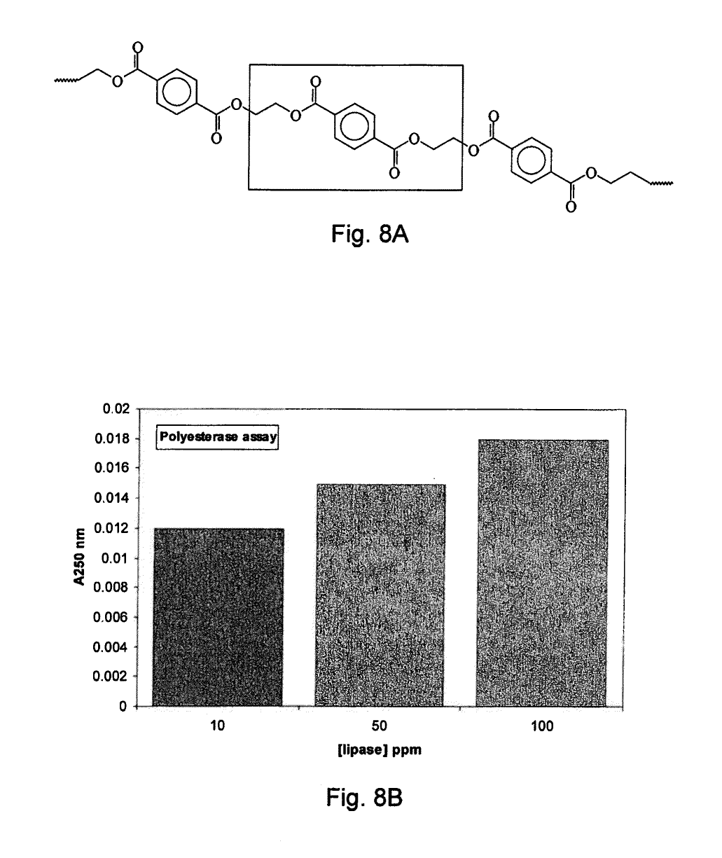

As further evidence for the versatility of the enzyme, Mgr-C also demonstrated carboxylic ester hydrolase activity against a polymer, i.e., PET polyester. As shown in FIG. 8B, Mgr-C hydrolyzed polyester to release terephthalate-containing fragments. Such experiments demonstrate the wide range of substrates that can be acted on by Mgr-C, supporting its use hydrolytic and synthetic reactions.

### V. Detergent Compositions Comprising an Mgr-C Polypeptide

An aspect of the compositions and methods is a detergent composition comprising an Mgr-C polypeptide (including a variant or fragment, thereof) and methods for using such compositions in cleaning applications. Cleaning applications include laundry or textile cleaning, dishwashing (manual and automatic), stain pre-treatment, and the like. Particular applications are those where lipids are a component of the soils or stains to be removed. Detergent compositions typically include an effective amount of Mgr-C or a variant thereof, e.g., at least 0.0001 weight percent, from about 0.0001 to about 1, from about 0.001 to about 0.5, from about 0.01 to about 0.1 weight percent, or even from about 0.1 to about 1 weight percent, or more.

In some embodiments, the detergent composition comprises a nonionic surfactants, in the absence of an ionic surfactant. Exemplary nonionic surfactants include but are not limited to polyoxyethylene esters of fatty acids, polyoxyethylene sorbitan esters (e.g., TWEENs), polyoxyethylene alcohols, polyoxyethylene isoalcohols, polyoxyethylene ethers (e.g., TRITONs and BRIJ), polyoxyethylene esters, polyoxyethylene-p-tert-octylphenols or octylphenyl-ethylene oxide condensates (e.g., NONIDET P40), ethylene oxide condensates with fatty alcohols (e.g., LUBROL), polyoxyethylene nonylphenols, polyalkylene glycols (SYNPERONIC F108), sugar-based surfactants (e.g., glycopyranosides, and thioglycopyranosides), and combinations and mixtures thereof. An exemplary surfactant is non-ionic ethoxylate surfactant found in DROPPS. In other embodiments, a small amount of ionic or zwitterionic detergent may be present, so long as it does not substantially interfere with Mgr-C polypeptide activity.

Detergent compositions may additionally include a detergent builder, a complexing agent, a polymer, a bleaching system, a stabilizer, a foam booster, a suds suppressor, an anti-corrosion agent, a soil-suspending agent, an anti-soil redeposition agent, a dye, a bactericide, a hydrotope, a tarnish inhibitor, an optical brightener, a fabric conditioner, and a perfume. The detergent compositions may also include enzymes, including but not limited to proteases, amylases, cellulases, lipases, or additional carboxylic ester hydrolases. The pH of the detergent compositions should be neutral to basic, as described, herein.

In using detergent compositions that include Mgr-C in cleaning applications, the fabrics, textiles, dishes, or other surfaces to be cleaned are incubated n the presence of the Mgr-C detergent composition for time sufficient to allow Mgr-C to hydrolyze lipids present in soil or stains, and then typically rinsed with water or another aqueous solvent to remove the Mgr-C detergent composition along with hydrolyzed lipids.

### VI. Mgr-C Polypeptides as Chemical Reagents

The preference of Mgr-C for short-chain lipids make the present polypeptides particularly useful for performing transesterification reactions involving C4-C10 substrates. Exemplary applications are the hydrolysis of milk fat; the synthesis of structured triglycerides, the synthesis and degradation of polymers, the formation of emulsifying agents and surfactants; the synthesis of ingredients for personal-care products, pharmaceuticals and agrochemicals, for making esters for use as perfumes and fragrances, for making biofuels and synthetic lubricants, for forming peracids, and for other uses in the oleochemical industry. Further uses for the above-described enzyme are described in U.S. Patent Pubs. 20070026106, 20060078648, and 20050196766, and in WO 2005/066347, which documents are incorporated by reference.

In general terms, a substrate and acceptor molecule are incubated in the presence of an Mgr-C polypeptide or variant thereof under conditions suitable for performing a transesterification reaction, followed by, optionally, isolating a product from the reaction. Alternatively, the conditions may in the context of a foodstuff and the product may become a component of the foodstuff without isolation.

Other aspects and embodiments of the present compositions and methods will apparent from the foregoing description and following example.

### EXAMPLES

The following examples are provided to demonstrate and illustrate certain preferred embodiments and aspects of the present invention and should not be construed as limiting.

### Example 1

### Assay Procedures

Various assays were used in the following Examples, as grouped together and set forth below for ease in reading. Any deviations from these protocols are specified in the particular Examples.

1. DGGR (1,2-O-Dilauryl-Rac-Glycero-3-Glutaric-Resorufin Ester) Assay to Determine Lipase/Esterase/Cutinase Activity

### Equipment:

Specrophotometer with temperature control capable of kinetic measurements

96-well microtiter plates

### Materials:

Assay buffer: 50 mM HEPES pH 8, 0.4 mg/ml MgCl₂, 1.2 mg/ml CaCl₂, 2% gum arabic Sigma, CAS 9000-01-5, catalog number G9752

Substrate: 664 μM 1,2-O-dilauryl-rac-glycero-3-glutaric-resorufin ester (DGGR) (Fluka 30058) dissolved in dimethylsulfoxide (DMSO, Pierce, 20688, water content <0.2%). A substrate solution was prepared by mixing of 4 parts of assay buffer and 1 part of substrate. A suitably diluted aliquot of T. reesei culture supernatant was added to 200 μl of the substrate solution in a 96-well microtiter plate. The hydrolysis of DGGR was monitored as a change of absorption at 572 nm that was followed in real time using a microtiter plate reader. The background rate (with no enzyme) was subtracted from the rate of the test samples.

2. Assay to Determine Carbon Chain-Length Preference

### Equipment:

Specrophotometer with temperature control capable of kinetic measurements

96-well microtiter plates

### Materials:

Assay buffer: 50 mM HEPES pH 6.0, 6 gpg, 3:1 Ca:Mg Hardness, 2% poly (vinyl) alcohol (PVA; Sigma 341584), 2% Triton X 100

The following substrates were used:

| Substrate | Abbr | Chain-length | Source |
|---|---|---|---|
| p-nitrophenyl | pNB | C4:0 | Sigma catalog no. N9876 (CAS |
| butyrate | | | 2635-84-9) |
| p-nitrophenyl | pNO | C8:0 | Alfa Aesar (Ward Hill, MA, USA), |
| octanoate | | | catalog no. L12022 |
| p-nitrophenyl | pND | C10:0 | Fluka catalog no. 21497 (CAS |
| decanoate | | | 1956-09-8) |
| p-nitrophenyl | pNP | C16:0 | Sigma, catalog no. N2752 (CAS |
| palmitate | | | 1492-30-4,) |
| p-nitrophenyl | pNS | C18:0 | Sigma, catalog no. N3502 (CAS |
| stearate | | | 104809-27-0) |

All substrates were dissolved in DMSO (Pierce, 20688, Water content <0.2%) and stored at −80° C. for long term storage. To measure lipase/esterase activity as a function of carbon chain length, each substrates was separately suspended in isopropanol to a concentration of 20 mM, and then diluted to 1 mM in assay buffer. 100 μL of each chain-length substrate in assay buffer was added to a 96-well microtiter plate and 10 μL of appropriately diluted enzyme was added to the substrate containing plate to initiate the reaction. The plate was immediately transferred to a plate reading spectrophotometer set at 25° C. (or different temperatures for determining temperature profile). The absorbance change in the kinetic mode was read for 5 minutes at 410 nm. The background rate (with no enzyme) was subtracted from the rate of the test samples.

3. Microtiter Plates Assay to Measure Triglyceride and Ester Hydrolysis

This assay was designed to measure the enzymatic release of fatty acids from triglyceride or ester substrates. The assay consisted of a hydrolysis reaction wherein incubation of enzyme with an emulsified substrate resulted in liberation of fatty acids, which were detected by direct measurement (e.g., HPLC) or by measurement of the reduction in turbidity of the emulsified substrate.

### Equipment:

Plate reading spectrophotometer capable of end point measurement (SpectraMax Plus384 (Molecular Devices, Sunnyvale, Calif., USA)

96-Well microtiter plates

Eppendorf Thermomixer

### Substrate:

Glycerol trioctanoate (Sigma, catalog no. T9126-100ML (CAS 538-23-8)

### Reagents:

NEFA (non-esterified fatty acid) assay reagent (HR Series NEFA-HR (2) NEFA kit, WAKO Diagnostics, Richmond, Va.)

### Procedure:

Emulsified triglycerides (0.75% (v/v or w/v)) were prepared in a buffer consisting of 50 mM HEPES pH 8.2, 6 gpg hardness and 2% gum arabic (Sigma, catalog number G9752 (CAS 9000-01-5). The solutions were mixed and sonicated for at least 2 minutes to prepare a stable emulsion. 200 μl of emulsified substrate was added to a 96-well microtiter plate and 20 μL of serially diluted enzyme samples were added to the substrate containing plate. The plate was covered with a plate sealer and incubated at 40° C. shaking for 1-2 hours. After incubation, the presence of fatty acids in solution was detected using the HR Series NEFA-HR (2) NEFA kit using the manufacturer's instructions. The NEFA kit measures the presence of non-esterified fatty acids.

4. Triglyceride Hydrolysis Microswatches Assay to Measure Lipase Activity

Microswatches treated with triglycerides were prepared as follows: EMPA 221 unsoiled cotton fabrics (Test Fabrics Inc. West Pittiston, Pa., USA) were cut to fit 96-well microtiter plates. 0.5-1 μl of neat trioctanoate or triolein was spotted on the microswatches, which were then left at room temperature for about 10 minutes. One triglyceride treated microswatch was placed in each well of a microtiter plate. A commercially available detergent composition or 50 mM HEPES pH 8.2, 6 gpg, 2% PVA (polyvinyl alcohol) was added to each well containing a microswatch. One commercially available detergent composition is DROPPS™ detergent (Laundry Dropps, Cot'n Wash Inc., Ardmore, Pa., USA), which contains a non-ionic ethoxylate surfactant and has relatively low water content (about 10% by weight). DROPPS™ was used at a final amount of 0.1%. Another commercially available detergent composition is TIDE® CW (Procter & Gamble, Cincinnati, Ohio, USA), which contains both anionic and nonionic surfactants and has relatively high water content (about 30-40% by weight). TIDE® CW was heat-inactivated prior to use to kill enzymes present in the composition.

10 μL of serially diluted enzyme sample was added to the wells. The plate was sealed with a plate sealer and incubated at 750 rpm at 40° C. for 60 minutes. After incubation, the supernatant was removed (and saved) from the swatches and the swatches were rinsed with 100 μL of detergent solution (the rinse was also saved) and blotted dry on paper towels. The presence of fatty acids in solution (i.e., the supernatant and rinse) and remaining on the cloth was detected using the HR Series NEFA-HR (2) NEFA kit (WAKO Diagnostics, Richmond, Va.) using the manufacturer's instructions.

5. Lard/Sudan Red 12-Well Applications Assay to Determine Cleaning Performance

1.5 cm mini-swatches cut from oily swatches (Technical stains of lard on cotton dyed with Sudan Red, STC CFT CS-62 Lard with Sudan Red, Test Fabrics, Inc., West Pittiston, Pa., USA) were pre-read using a CROMA METER CR-200 Minolta reflectometer. One ml reactions were performed in the buffer (50 mM HEPES pH 8.2, 6 gpg 0.1% DROPPS™ detergent) with concentrations of Mgr-C ranging from 0 to 10 ppm. The reactions were incubated at 20° C. for 20 minutes. After incubation, the 1.5 cm mini-swatches were washed with distilled water and dried for 30 minutes at 60° C. Cleaning was calculated as the difference of the post- and pre-cleaning reflectometery measurements for each swatch and is reported as change in overall reflectance (i.e., deltaE).

### Example 2

### Identification, Cloning and Expression of Mgr-C Cutinase (abH36.1)

The presumptive Magnaporthe grisea cutinase cutinase gene (mgr-C) was identified in the genomic sequence of the rice blast fungus Magnaporthe grisea strain 70-15 (Dean, R. A. et al. (2005) Nature 434:980-986; PubMed Acc No. XP_{—}365241). The predicted amino acid sequence of M. grisea cutinase (Mgr-C) is listed as SEQ ID NO. 1.

SEQ ID NO 1:
Predicted amino acid sequence of M. grisea
cutinase protein (Mgr-C)

| | |
|---|---|
| 1 | MQFITVALTL IALASASPIA TNVEKPSELE ARQLNSVRND |
| | LISGNAAACP |
| 51 | SVILIFARAS GEVGNMGLSA GTNVASALER EFRNDIWVQG |
| | VGDPYDAALS |
| 101 | PNFLPAGTTQ GAIDEAKRMF TLANTKCPNA AVVAGGYSQG |
| | TAVMFNAVSE |
| 151 | MPAAVQDQIK GVVLFGYTKN LQNRGRIPDF PTEKTEVYCN |
| | ASDAVCFGTL |
| 201 | FLLPAHFLYT TESSIAAPNW LIRQIRAA |

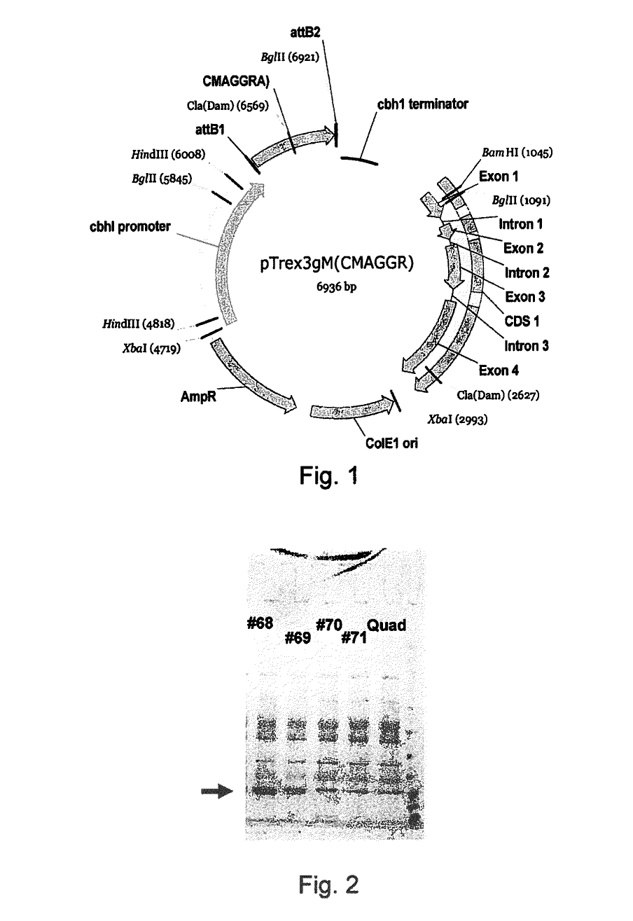

An artificial mgr-C gene codon-optimized for expression in Trichoderma reesei was synthesized by DNA 2.0 (Menlo Park, Calif., USA) and placed under control of cbhI promoter of T. reesei in the vector pTrex3gM (FIG. 1). From the resulting construct a 5.2 kb SfiI-SfiI fragment was excised and used to transform the spores of T. reesei quad-deleted strain (Δcbh1, Δcbh2, Δegl1, Δegl2, described in WO05/001036) by electroporation (WO28153712A2). The complete sequence of the transforming DNA fragment is listed as SEQ ID NO 2.

| SEQ ID NO 2: | |
|---|---|
| Nucleotide sequence of expression cassette containing | |
| M. grisea cutinase gene used to transform T reesei | |
| 1 | AGGCCTCTAG AGTTGTGAAG TCGGTAATCC CGCTGTATAG TAATACGAGT CGCATCTAAA |
| 61 | TACTCCGAAG CTGCTGCGAA CCCGGAGAAT CGAGATGTGC TGGAAAGCTT CTAGCGAGCG |
| 121 | GCTAAATTAG CATGAAAGGC TATGAGAAAT TCTGGAGACG GCTTGTTGAA TCATGGCGTT |
| 181 | CCATTCTTCG ACAAGCAAAG CGTTCCGTCG CAGTAGCAGG CACTCATTCC CGAAAAAACT |
| 241 | CGGAGATTCC TAAGTAGCGA TGGAACCGGA ATAATATAAT AGGCAATACA TTGAGTTGCC |
| 301 | TCGACGGTTG CAATGCAGGG GTACTGAGCT TGGACATAAC TGTTCCGTAC CCCACCTCTT |
| 361 | CTCAACCTTT GGCGTTTCCC TGATTCAGCG TACCCGTACA AGTCGTAATC ACTATTAACC |
| 421 | CAGACTGACC GGACGTGTTT TGCCCTTCAT TTGGAGAAAT AATGTCATTG CGATGTGTAA |
| 481 | TTTGCCTGCT TGACCGACTG GGGCTGTTCG AAGCCCGAAT GTAGGATTGT TATCCGAACT |
| 541 | CTGCTCGTAG AGGCATGTTG TGAATCTGTG TCGGGCAGGA CACGCCTCGA AGGTTCACGG |
| 601 | CAAGGGAAAC CACCGATAGC AGTGTCTAGT AGCAACCTGT AAAGCCGCAA TGCAGCATCA |
| 661 | CTGGAAAATA CAAACCAATG GCTAAAAGTA CATAAGTTAA TGCCTAAAGA AGTCATATAC |
| 721 | CAGCGGCTAA TAATTGTACA ATCAAGTGGC TAAACGTACC GTAATTTGCC AACGGCTTGT |
| 781 | GGGGTTGCAG AAGCAACGGC AAAGCCCCAC TTCCCCACGT TTGTTTCTTC ACTCAGTCCA |
| 841 | ATCTCAGCTG GTGATCCCCC AATTGGGTCG CTTGTTTGTT CCGGTGAAGT GAAAGAAGAC |
| 901 | AGAGGTAAGA ATGTCTGACT CGGAGCGTTT TGCATACAAC CAAGGGCAGT GATGGAAGAC |
| 961 | AGTGAAATGT TGACATTCAA GGAGTATTTA GCCAGGGATG CTTGAGTGTA TCGTGTAAGG |
| 1021 | AGGTTTGTCT GCCGATACGA CGAATACTGT ATAGTCACTT CTGATGAAGT GGTCCATATT |
| 1081 | GAAATGTAAA GTCGGCACTG AACAGGCAAA AGATTGAGTT GAAACTGCCT AAGATCTCGG |
| 1141 | GCCCTCGGGC CTTCGGCCTT TGGGTGTACA TGTTTGTGCT CCGGGCAAAT GCAAAGTGTG |
| 1201 | GTAGGATCGA ACACACTGCT GCCTTTACCA AGCAGCTGAG GGTATGTGAT AGGCAAATGT |
| 1261 | TCAGGGGCCA CTGCATGGTT TCGAATAGAA AGAGAAGCTT AGCCAAGAAC AATAGCCGAT |
| 1321 | AAAGATAGCC TCATTAAACG GAATGAGCTA GTAGGCAAAG TCAGCGAATG TGTATATATA |
| 1381 | AAGGTTCGAG GTCCGTGCCT CCCTCATGCT CTCCCCATCT ACTCATCAAC TCAGATCCTC |
| 1441 | CAGGAGACTT GTACACCATC TTTTGAGGCA CAGAAACCCA ATAGTCAACC ATCACAAGTT |
| 1501 | TGTACAAAAA AGCAGGCTAT GCAGTTTATT ACCGTCGCTT TGACATTGAT TGCCTTGGCT |
| 1561 | TCCGCCTCGC CTATTGCCAC TAACGTGGAG AAGCCCTCTG AGCTGGAAGC TCGCCAGCTT |
| 1621 | AACTCGGTCC GCAATGACCT GATCAGCGGC AACGCAGCTG CCTGCCCCTC GGTCATTCTG |
| 1681 | ATCTTCGCCC GCGCCTCTGG TGAGGTCGGA AACATGGGAC TCTCCGCCGG TACCAACGTC |
| 1741 | GCCTCGGCTT TGGAGCGTGA GTTTCGCAAC GATATTTGGG TCCAGGGCGT CGGCGATCCT |
| 1801 | TACGACGCCG CTCTCTCACC AAACTTCCTG CCTGCTGGCA CCACCCAGGG CGCCATCGAT |
| 1861 | GAGGCCAAGC GCATGTTCAC ACTGGCCAAC ACGAAGTGCC CCAACGCTGC TGTTGTTGCC |
| 1921 | GGCGGATACT CGCAGGGCAC GGCCGTTATG TTCAACGCGG TTAGCGAGAT GCCCGCAGCT |
| 1981 | GTGCAGGACC AGATCAAGGG AGTGGTTCTG TTCGGCTACA CGAAGAACCT CCAGAACCGC |
| 2041 | GGACGCATCC CGGACTTTCC GACCGAAAAG ACTGAGGTCT ACTGCAACGC TTCGGACGCG |
| 2101 | GTGTGCTTTG GTACGCTGTT CCTCCTTCCT GCGCACTTCC TCTATACTAC CGAGTCGAGC |
| 2161 | ATCGCCGCTC CGAACTGGTT GATTCGTCAA ATCCGCGCTG CGTAATGAGA TCTACCCAGC |
| 2221 | TTTCTTGTAC AAAGTGGTGA TCGCGCCGCG CGCCAGCTCC GTGCGAAAGC CTGACGCACC |
| 2281 | GGTAGATTCT TGGTGAGCCC GTATCATGAC GGCGGCGGGA GCTACATGGC CCCGGGTGAT |
| 2341 | TTATTTTTTT TGTATCTACT TCTGACCCTT TTCAAATATA CGGTCAACTC ATCTTTCACT |
| 2401 | GGAGATGCGG CCTGCTTGGT ATTGCGATGT TGTCAGCTTG GCAAATTGTG GCTTTCGAAA |
| 2461 | ACACAAAACG ATTCCTTAGT AGCCATGCAT TTTAAGATAA CGGAATAGAA GAAAGAGGAA |
| 2521 | ATTAAAAAAA AAAAAAAAAC AAACATCCCG TTCATAACCC GTAGAATCGC CGCTCTTCGT |
| 2581 | GTATCCCAGT ACCAGTTTAT TTTGAATAGC TCGCCCGCTG GAGAGCATCC TGAATGCAAG |
| 2641 | TAACAACCGT AGAGGCTGAC ACGGCAGGTG TTGCTAGGGA GCGTCGTGTT CTACAAGGCC |
| 2701 | AGACGTCTTC GCGGTTGATA TATATGTATG TTTGACTGCA GGCTGCTCAG CGACGACAGT |
| 2761 | CAAGTTCGCC CTCGCTGCTT GTGCAATAAT CGCAGTGGGG AAGCCACACC GTGACTCCCA |
| 2821 | TCTTTCAGTA AAGCTCTGTT GGTGTTTATC AGCAATACAC GTAATTTAAA CTCGTTAGCA |
| 2881 | TGGGGCTGAT AGCTTAATTA CCGTTTACCA GTGCCGCGGT TCTGCAGCTT TCCTTGGCCC |
| 2941 | GTAAAATTCG GCGAAGCCAG CCAATCACCA GCTAGGCACC AGCTAAACCC TATAATTAGT |
| 3001 | CTCTTATCAA CACCATCCGC TCCCCCGGGA TCAATGAGGA GAATGAGGGG GATGCGGGGC |
| 3061 | TAAAGAAGCC TACATAACCC TCATGCCAAC TCCCAGTTTA CACTCGTCGA GCCAACATCC |
| 3121 | TGACTATAAG CTAACACAGA ATGCCTCAAT CCTGGGAAGA ACTGGCCGCT GATAAGCGCG |
| 3181 | CCCGCCTCGC AAAAACCATC CCTGATGAAT GGAAAGTCCA GACGCTGCCT GCGGAAGACA |
| 3241 | GCGTTATTGA TTTCCCAAAG AAATCGGGGA TCCTTTCAGA GGCCGAACTG AAGATCACAG |
| 3301 | AGGCCTCCGC TGCAGATCTT GTGTCCAAGC TGGCGGCCGG AGAGTTGACC TCGGTGGAAG |
| 3361 | TTACGCTAGC ATTCTGTAAA CGGGCAGCAA TCGCCCAGCA GTTAGTAGGG TCCCCTCTAC |
| 3421 | CTCTCAGGGA GATGTAACAA CGCCACCTTA TGGGACTATC AAGCTGACGC TGGCTTCTGT |
| 3481 | GCAGACAAAC TGCGCCCACG AGTTCTTCCC TGACGCCGCT CTCGCGCAGG CAAGGGAACT |
| 3541 | CGATGAATAC TACGCAAAGC ACAAGAGACC CGTTGGTCCA CTCCATGGCC TCCCCATCTC |
| 3601 | TCTCAAAGAC CAGCTTCGAG TCAAGGTACA CCGTTGCCCC TAAGTCGTTA GATGTCCCTT |
| 3661 | TTTGTCAGCT AACATATGCC ACCAGGGCTA CGAAACATCA ATGGGCTACA TCTCATGGCT |
| 3721 | AAACAAGTAC GACGAAGGGG ACTCGGTTCT GACAACCATG CTCCGCAAAG CCGGTGCCGT |
| 3781 | CTTCTACGTC AAGACCTCTG TCCCGCAGAC CCTGATGGTC TGCGAGACAG TCAACAACAT |
| 3841 | CATCGGGCGC ACCGTCAACC CACGCAACAA GAACTGGTCG TGCGGCGGCA GTTCTGGTGG |
| 3901 | TGAGGGTGCG ATCGTTGGGA TTCGTGGTGG CGTCATCGGT GTAGGAACGG ATATCGGTGG |
| 3961 | CTCGATTCGA GTGCCGGCCG CGTTCAACTT CCTGTACGGT CTAAGGCCGA GTCATGGGCG |
| 4021 | GCTGCCGTAT GCAAAGATGG CGAACAGCAT GGAGGGTCAG GAGACGGTGC ACAGCGTTGT |
| 4081 | CGGGCCGATT ACGCACTCTG TTGAGGGTGA GTCCTTCGCC TCTTCCTTCT TTTCCTGCTC |
| 4141 | TATACCAGGC CTCCACTGTC CTCCTTTCTT GCTTTTTATA CTATATACGA GACCGGCAGT |
| 4201 | CACTGATGAA GTATGTTAGA CCTCCGCCTC TTCACCAAAT CCGTCCTCGG TCAGGAGCCA |
| 4261 | TGGAAATACG ACTCCAAGGT CATCCCCATG CCCTGGCGCC AGTCCGAGTC GGACATTATT |
| 4321 | GCCTCCAAGA TCAAGAACGG CGGGCTCAAT ATCGGCTACT ACAACTTCGA CGGCAATGTC |
| 4381 | CTTCCACACC CTCCTATCCT GCGCGGCGTG GAAACCACCG TCGCCGCACT CGCCAAAGCC |
| 4441 | GGTCACACCG TGACCCCGTG GACGCCATAC AAGCACGATT TCGGCCACGA TCTCATCTCC |
| 4501 | CATATCTACG CGGCTGACGG CAGCGCCGAC GTAATGCGCG ATATCAGTGC ATCCGGCGAG |
| 4561 | CCGGCGATTC CAAATATCAA AGACCTACTG AACCCGAACA TCAAAGCTGT TAACATGAAC |
| 4621 | GAGCTCTGGG ACACGCATCT CCAGAAGTGG AATTACCAGA TGGAGTACCT TGAGAAATGG |
| 4681 | CGGGAGGCTG AAGAAAAGGC CGGGAAGGAA CTGGACGCCA TCATCGCGCC GATTACGCCT |
| 4741 | ACCGCTGCGG TACGGCATGA CCAGTTCCGG TACTATGGGT ATGCCTCTGT GATCAACCTG |
| 4801 | CTGGATTTCA CGAGCGTGGT TGTTCCGGTT ACCTTTGCGG ATAAGAACAT CGATAAGAAG |
| 4861 | AATGAGAGTT TCAAGGCGGT TAGTGAGCTT GATGCCCTCG TGCAGGAAGA GTATGATCCG |
| 4921 | GAGGCGTACC ATGGGGCACC GGTTGCAGTG CAGGTTATCG GACGGAGACT CAGTGAAGAG |
| 4981 | AGGACGTTGG CGATTGCAGA GGAAGTGGGG AAGTTGCTGG GAAATGTGGT GACTCCATAG |
| 5041 | CTAATAAGTG TCAGATAGCA ATTTGCACAA GAAATCAATA CCAGCAACTG TAAATAAGCG |
| 5101 | CTGAAGTGAC CATGCCATGC TACGAAAGAG CAGAAAAAAA CCTGCCGTAG AACCGAAGAG |
| 5161 | ATATGACACG CTTCCATCTC TCAAAGGAAG AATCCCTTCA GGGTTGCGTT TCCAGTCTAG |
| 5221 | AGGCCATTT |

Transformants were selected on a medium containing acetamide as the sole source of nitrogen (acetamide 0.6 g/l; cesium chloride 1.68 g/l; glucose 20 g/l; potassium dihydrogen phosphate 15 g/l; magnesium sulfate heptahydrate 0.6 g/l; calcium chloride dihydrate 0.6 g/l; iron (II) sulfate 5 mg/l; zinc sulfate 1.4 mg/l; cobalt (II) chloride 1 mg/1; manganese (II) sulfate 1.6 mg/l; agar 20 g/l; pH 4.25). Transformed colonies appeared in about 1 week.

Individual transformants were transferred onto fresh acetamide selective plates and allowed to grow for 2-4 days. Isolates showing stable growth on selective medium were used to inoculate 0.2 ml of lactose defined medium (WO 2005/001036) in the well of a microtiter plate equipped with a microfilter at the bottom (Millipore MULTISCREEN-GV™). The plates were incubated for 4-5 days at 25-28° C. in an atmosphere of pure oxygen.

The culture media were separated by filtration and analyzed by polyacrylamide gel electrophoresis in the presence of sodium dodecylsulfate (SDS PAGE). The results of this analysis demonstrated the appearance of a new protein band of a molecular weight corresponding to the mature Mgr-C cutinase polypeptide (FIG. 2). Lanes marked #68 to #71, were loaded with culture supernatants from each of four different transformants cultivated in an expression inducing medium in a microtiter plate. The lane marked “Quad” was loaded with similarly prepared culture supernatant from control cells.

All transformants that showed a new protein band on SDS-PAGE also had a high level of activity in DGGR assay described in Example 1. Essentially no activity was detected in the culture supernatants of the control (untransformed) strain of T. reesei.

Four two-liter shake flasks each containing about 300 ml of lactose defined medium were inoculated with the spores of transformant #68 that had displayed high level of Mgr-C cutinase production in microtiter plate experiments. The flasks were placed onto a rotary shaker set at 200 rpm and 28° C. for 6 days. The culture supernatants were separated from the mycelium by filtration through four layers of MIRACLOTH® (Calbiochem), concentrated by ultra-filtration using a 10 kDa membrane, and, finally, clarified by high speed centrifugation (14,000 rpm in Sorvall SS-34 rotor). The resulting preparation was used for subsequent characterization of Mgr-C cutinase.

### Example 3

### pH Profile of Mgr-C Cutinase

In this example, the pH profile of Mgr-C was studied across a range of pH values (i.e., pH 4-pH 10). Mgr-C activity at different pH values was measured in 25 mM Universal pH buffer, 1% PVA, 1% TritonX 100 at 25° C. using 1 mM pNO as substrate as described in Example 1. The results are reported as activity of Mgr-C at various pH values relative to activity measured at pH 6 and are shown in FIG. 3.

### Example 4

### Temperature Profile of Mgr-C Cutinase

In this example, the effect of temperature on the activity of Mgr-C was studied across a range of temperatures (10° C.-90° C.). Mgr-C activity was measured as release of p-nitrophenylate from pNO (a C8:0 substrate) as a function of temperature, using the procedure described in the chain-length dependence assay in Example 1. Results are shown in FIG. 4. Mgr-C had maximal activity between 20° C. and 40° C.

### Example 5

### Assessment of Hydrolytic Activity of Mgr-C Cutinase

In this example, the ability of Mgr-C cutinase to hydrolyse synthetic substrates and triglycerides was tested using assays described in Example 1.

A. Hydrolysis of p-Nitrophenyl Esters of Different Chain-Lengths

Chain length preference of Mgr-C was studied by measuring hydrolysis activity of the enzyme as a function of chain-length. 10 μL of serially diluted enzyme sample was incubated with 100 μL of p-nitrophenyl ester substrates (i.e., C4, C8, C10, C16, and C18 substrates) in reaction buffer at pH 6, as described in Example 1. The release of p-nitrophenylate product was kinetically measured using the assay described in Example 1. The rate of product release obtained using each substrate was normalized to the highest activity. The results are shown in FIG. 5.

B. Hydrolysis of Triglycerides

10 μL aliquots of serially diluted enzyme samples were incubated with trioctanoate (0.75%) in a 2% gum arabic emulsion in the buffer containing 50 mM HEPES, pH 8.2, 6 gpg, 2% PVA at 40° C., 450 rpm for 2 hours. The release of products was measured using the triglyceride hydrolysis assay to determine lipase activity in 96-well microtiter plates, as described in Example 1. Trioctanoate hydrolysis in solution is reported as percent relative activity (activity normalized to activity measured with 14 μg/mL lipase) and is shown in FIG. 6.

### Example 6

### Hydrolysis of Triglycerides on Cloth

This assay simulates cleaning performance of the Mgr-C enzyme. Increasing amounts of Mgr-C were tested for the ability to hydrolyse trioctanoate bound to cloth in buffer containing 2% PVA or detergent (0.1% DROPPS) using the triglyceride hydrolysis assay on microswatches assay described in Example 1. The results were reported as percent relative activity (i.e., activity normalized to activity measured with 0.1 μg/mL lipase in buffer only). FIG. 7A shows the amounts of free fatty acids remaining on cloth and FIG. 7B shows the amounts of free fatty acids released into solution.

### Example 7

### Polyesterase Activity of Mgr-C Cutinase

In this example, Mgr-C was assayed for polyesterase activity using a spectrophotometric assay. The assay monitors the release of soluble terephthalate-containing fragments resulting from the enzymatic hydrolysis of insoluble polyester (polyethylene terephthalate, PET) (FIG. 8A). These fragments have a strong absorbance at around 240-250 nm (ε_{M}˜10,000).

The substrate PET was obtained from Scientific Polymer Products (catalog no. 138). The assay buffer used was 50 mM HEPES pH 8.2+0.01% BRIJ® 35 (Sigma 16013). For assaying for polyesterase activity, PET substrate was diluted to 90 mg/ml in assay buffer in 1.5 ml tubes. Mgr-C was diluted to 10, 50 and 100 ppm in the substrate containing tubes. Control samples with enzyme and no substrate and with substrate and no enzyme were also prepared. All samples were incubated at 40° C. with 1000 rpm of shaking in thermomixer for 6 hours. At the end of the reaction, 80 μL from each tube was transferred to a 96-well microtiterplate for spectrophotometric measurement. Absorbance was measured at 250 nm. The background absorbance (from wells lacking substrate) was subtracted from the corresponding test samples (wells containing substrate). The results are shown in FIG. 8B, and demonstrate that Mgr-C has polyesterase activity.

### Example 8

### Lard/Sudan Red 12-Well Applications Assay to Determine Cleaning Performance of Mgr-C Cutinase

In this Example, the cleaning performance of Mgr-C was measured using microswatches stained with oily soils in a 12-well plate format as described in Example 1 (Lard/Sudan Red 12-well applications assay to determine cleaning performance). The results are shown in FIG. 9. Mgr-C exhibited cleaning activity on lard/Sudan red swatches at reduced temperature (20° C.).

1-12. (canceled)

13. A detergent composition comprising a recombinant Mgr-C polypeptide of.

14. The composition of claim 13, comprising a non-ionic surfactant.

15. The composition of claim 14, wherein the surfactant is non-ionic ethoxylate surfactant.

16. A method for hydrolyzing a lipid present in a soil or stain on a surface, comprising contacting the surface with a detergent composition comprising a recombinant Mgr-C polypeptide and a non-ionic surfactant.

17. The method of claim 16, wherein the non-ionic surfactant is an ethoxylate surfactant.

18. A method for performing a transesterification reaction comprising contacting a donor molecule with a composition comprising a recombinant Mgr-C polypeptide.

19. The method of claim 18, wherein the donor molecule has a C4-C10 carbon chain.

20. (canceled)

21. The method of claim 16, wherein the Mgr-C polypeptide has an amino acid sequence that is at least 80% identical to the amino acid sequence of SEQ ID NO: 1.

22. The method of claim 21, wherein the Mgr-C polypeptide has an amino acid sequence that is at least 90% identical to the amino acid sequence of SEQ ID NO: 1.

23. The method of claim 21, wherein the Mgr-C polypeptide has an amino acid that is substantially identical to amino acid sequence of SEQ ID NO: 1.

24. The method of claim 21, wherein the Mgr-C polypeptide has the amino acid sequence of SEQ ID NO: 1.

25. The detergent composition of claim 13, further comprising a hydrolytic enzyme.

26. The detergent composition of claim 13, further comprising a solubilizer.

27. The detergent composition of claim 13, further comprising a bleaching agent.

28. The detergent composition of claim 13, further comprising an antioxidant.

29. The detergent composition of claim 13, further comprising a builder.

30. The detergent composition of claim 13, further comprising a fluorescent dye.
